(19) United States
(12) Patent Application Publication
(10) Pub. No.: US 2004/0087590 A1
(43) Pub. Date: May 6, 2004

(54) Novel biphenyl and biphenyl-like cannabinoids

(75) Inventors: Alexandros Makriyannis, Mystic, CT (US); Xin-Zhong Lai, Storrs, CT (US); Dai Lu, Storrs, CT (US)

Correspondence Address: ALIX YALE & RISTAS LLP, 750 MAIN STREET, SUITE 1400, HARTFORD, CT, 06103, US

(73) Assignee: University of Connecticut, Farmington, CT

(21) Appl. No.: 10/647,550

(22) Filed: Aug. 25, 2003

Related U.S. Application Data
(60) Provisional application No. 60/405,608, filed on Aug. 23, 2002.

(51) Int. Cl. A61K 31/501; A61K 31/497; A61K 31/4965; C 07D 4 3/02

(52) U.S. Cl. 514/252.03; 514/255.05; 514/256; 514/336; 544/238; 544/333; 544/405; 546/268.1

(57) ABSTRACT

Novel biphenyl and biphenyl-like cannabinoid compounds are presented. These compounds, when administered in a therapeutically effective amount to an individual or animal, result in a sufficiently high level of that compound in the individual or animal to cause a physiological response. The physiological response useful to treat a number of physiological conditions.

### CROSS REFERENCE TO RELATED APPLICATIONS

This application claims the benefit of U.S. Provisional Application No. 60/405,608, filed Aug. 23, 2002, the contents of each of which are incorporated by reference in their entirety.

### STATEMENT REGARDING FEDERALLY SPONSORED RESEARCH OR DEVELOPMENT

This invention was made with Government support under Contract No. DA3801 awarded by the National Institute of Health. The Government has certain rights in the invention.

### FIELD OF THE INVENTION

The present invention relates generally to cannabinoid analogs. The invention is more particularly concerned with new and improved biphenyl cannabinoids and the derivative biphenyl-like cannabinoids. In some embodiments the novel compounds exhibit high binding affinities for the CB1 or CB2 cannabinoid receptor. Another aspect of the invention comprises pharmaceutical preparations employing these analogs. A further aspect of the invention comprises a method of administering therapeutically effective amounts of the analogs to provide a physiological effect.

### BACKGROUND OF THE INVENTION

The active components of Cannabis Sativa, or Marijuana, are known to exert behavioral and psychotropic effects but also possess therapeutic properties in a variety of areas such as the central nervous system, the cardiovascular system, the immune system and endocrine system [Kumar R N, et al, Pharmacological actions and therapeutic uses of cannabis and cannabinoids, Anesthesia, 2001, 56: 1059-1068]. The therapeutic applications of most of active cannabinoids are strongly limited by their addictive and psychotropic properties [Nahas G, Marijuana and Medicine; 1999, Human Press Inc., Totowa, N.J.].

Representative classical cannabinoid (−)-Δ⁹-Tetrahydrocannabinol (Δ⁹-THC) is the major active constituent extracted from Cannabis Sativa (Marijuana). The pharmacological effects of cannabinoids pertain to a variety of areas such as the central nervous system, the cardiovascular system, the immune system and endocrine system. Most of the effects of cannabinoids are due to an interaction with specific high-affinity receptors. Presently, two cannabinoid receptors have been characterized: CB1, a central receptor found in the mammalian brain and a number of other sites in the peripheral tissues and CB2, a peripheral receptor found principally in cells related to the immune system. Characterization of these receptors has been made possible by the development of specific synthetic ligands such as the agonists WIN 55212-2 (aminoalkyl indole) and CP 55,940 (non-classic cannabinoid).

In addition to acting at the cannabinoid receptors, cannabinoids such as Δ⁹-THC also affect cellular membranes, thereby producing undesirable side effects such as drowsiness, impairment of monoamine oxidase function and impairment of non-receptor mediated brain function. The therapeutic applications of most naturally occurring cannabinoids are limited by their psychotropic properties [Nahas G, Marijuana and Medicine; 1999, Human Press Inc., Totowa, N.J.].

The CB1 cannabinoid receptor has been detected in the central nervous system (CNS) and in certain peripheral tissues including pituitary gland, immune cells, reproductive organs, gastrointestinal tissues, superior cervical ganglion, heart, lung, urinary bladder and adrenal gland [Pertwee R G. Pharmacology of cannabinoid CB1 and CB2 receptors. Pharmacol Ther. 1997;74(2):129-80]. The highest expression of CB1 receptors is found in human brain, particularly in cerebellum. The central distribution pattern of CB1 receptors accounts for several prominent pharmacological properties of cannabinoids, such as impairing cognition and memory and alternating the control of motor function, and mediating the psychotropic effects and other neurobehavioral effects of cannabinoids. CB1 receptors are also found on pain pathways in brain, spinal cord and at the peripheral terminals of primary sensory neurons [a) Rice A S. Cannabinoids and pain. Curr Opin Investig Drugs. 2001 March;2(3):399-414; b) Campbell F A et al, Are cannabinoids an effective and safe treatment option in the management of pain? A qualitative systematic review. BMJ. 2001 Jul. 7;323(7303):13-6.], with the latter two presenting attractive targets for separating the analgesic and psychotropic effects of cannabinoids. Conversely, the CB2 cannabinoid receptor does not appear to be expressed within the CNS but is the predominant form of the cannabinoid receptor expressed within immune system. Significant presence of CB2 receptor has been detected in human tonsils, leukocytes, and spleen [Galiegue S et al. Expression of central and peripheral cannabinoid receptors in human immune tissues and leukocyte subpopulations. Eur J Biochem. 1995 Aug. 15;232(1):54-61]. In human leukocytes, CB2 receptors were found with particularly high concentration in B-cells, natural killer cells and macrophage. The significant and predominant presence of cannabinoid receptor CB2 subtype in immune system suggest that CB2 receptor could be the most likely cannabinoid receptor that mediates the immunomodulatory effects of cannabinoids. The immune modulatory effects of cannabinoids are considerably broad, such as altering immune cell proliferation and function, altering antibody formation and altering cytokine production [a) Kaminski N E et al, Cannabinoid receptors CB1 and CB2: a characterization of expression and adenylate cyclase modulation within the immune system. Toxicol Appl Pharmacol. 1997 February; 142(2):278-87; b) Berdyshev E V, Cannabinoid receptors and the regulation of immune response. Chem Phys Lipids. 2000 November; 108(1-2):169-90; c) Kaminski N E, Regulation of the cAMP cascade, gene expression and immune function by cannabinoid receptors. J Neuroimmunol. 1998 Mar. 15;83(1-2):124-32; d) Klein T W et al, The cannabinoid system and cytokine network. Proc Soc Exp Biol Med. 2000 October; 225(1):1-8; e) Klein T W et al, Cannabinoid receptors and immunity. Immunol Today. 1998 August; 19(8):373-81; f) Cannabinoid receptors and the regulation of immune response. Chem Phys Lipids. 2000 November; 108(1-2):169-90].

The discovery of cannabinoid receptors was followed by the demonstration of the existence of endogenous cannabinoid receptor agonists such as arachidonoylethanolamide (anandamide) and 2-arachidonoylglycerol (2-AG) [Maccarron M., Endocannabinoids and their actions. Vitamins and Hormones 2002;65:225-255]. There is evidence that both these compounds can serve as neuromodulators or neurotransmitters. Biological organization of the endogenous cannabinoid system includes the CB1 and CB2 receptors, their endogenous ligands and the multiple metabolic pathways for the synthesis, degradation and reuptake of the endogenous ligands. Both anandamide and 2-AG are synthesized by neurones on demand. They can undergo depolarization-induced release from neurons. After their release and interaction with the receptors, they are rapidly removed from the extracellular space by a membrane transport process yet to be fully characterized [Beltramo M., Functional role of high-affinity anandamide transport, as revealed by selective inhibition. Science 1997; 277(5329):1094-1097]. Once within the cell, anandamide is hydrolysed to arachidonic acid and ethanolamine by the microsomal enzyme, fatty acid amide hydrolase (FAAH). The amplitude and duration of fatty acid amide signals can be regulated in vivo primarily by this integral membrane protein. Recently, the crystal structure of this enzyme was reported [Bracey, M et al, Structural Adaptations in a Membrane Enzyme That Terminates Endocannabinoid Signaling. Science 2002; 298(5599): 1793-1796.]. 2-AG can also be hydrolyzed enzymatically, both by fatty acid amide hydrolase (FAAH) and by monoacylglycerol (MAG) lipase [Ueda, N., Endocannabinoid hydrolases. Prostaglandins & Other Lipid Mediators 2002;68-69:521-534.].

### SUMMARY OF THE INVENTION

Briefly stated, one aspect of the present invention comprises novel biphenyl cannabinoids and the derivative biphenyl-like cannabinoids. Some of the inventive compounds are a group of potent cannabimimetic ligands possessing high cannabinoid receptor affinity and CB2 receptor selectivity. Compared to the classical cannabinoids and the endogenous cannabinoid receptor ligands anandamide and 2-arachidonyl glycerol, some of the biphenyl compounds and the derivative biphenyl-like cannabinoids are more potent, more stable and easier to prepare. Some of the compounds also possess considerable selectivity mostly for the CB2 receptor.

The compounds described in this invention are generally represented by compound formula I.

One embodiment of the invention comprises compound formula I

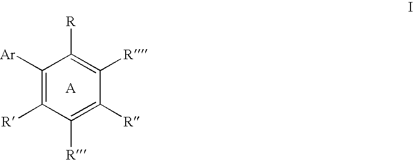

The “A” ring atoms of compound formula I comprise carbon and 0 to 2 nitrogen heteroatoms.

Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group.

R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,

E₁ and E₂ are each independently H or alkyl.

R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,

E₁ and E₂ are each independently H or alkyl.

R″, R′″ and R″″ each independently comprises Y-D₁-D₂-T₂, H, halogen, alkyl, alkoxy or a substituent group as defined later.

Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms.

D₁ is optionally present and if present comprises alkyl,

D₂ comprises H, alkyl, NH, N-alkyl, O-alkyl, S-alkyl, a carbocyclic ring, a bicyclic, a tricyclic ring, an aromatic or heteroaromatic ring,

T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group as defined later;

In one variation advantageous for cannabimimetic activity only one of R″, R′″ and R″″ comprises Y-D₁-D₂-T₂ and the others of R″, R′″ and R″″ each independently comprise H, halogen, alkyl, alkoxy or a substituent group as defined later.

In another variation advantageous for cannabimimetic activity:

R′″ comprises H, halogen, C(halogen)₃, lower alkyl or alkoxy;

R″″ comprises H, halogen, C(halogen)₃, lower alkyl or alkoxy; and

R″ comprises —Y-D₁-D₂-T₂,

Y comprises C(CH₃)₂, CH₂ or CH(CH₃),

D₁ is optionally present and if present comprises alkyl,

D₂ comprises H, an alkyl, NH, N-alkyl, O-alkyl, S-alkyl, a carbocyclic ring, a bicyclic ring, a tricyclic ring, an aromatic ring or a heteroaromatic ring,

T₂ is optionally present and if present comprises an aromatic ring, a heteroaromatic ring, a heterocyclic ring, H, OH, halogen or a substituent group.

In another variation advantageous for cannabimimetic activity:

R′″ comprises H, halogen, C(halogen)₃, lower alkyl or alkoxy;

R″″ comprises H, halogen, C(halogen)₃, lower alkyl or alkoxy; and

R″ comprises —Y-D₁-D₂-T₂,

Y comprises O, NH or N-alkyl,

D₁ is optionally present and if present comprises alkyl,

D₂ comprises H, an alkyl, NH, N-alkyl, O-alkyl, S-alkyl, a carbocyclic ring, a bicyclic ring, a tricyclic ring, an aromatic ring or a heteroaromatic ring,

T₂ is optionally present and if present comprises an aromatic ring, a heteroaromatic ring, a heterocyclic ring, H, OH, halogen or a substituent group.

In another variation advantageous for cannabimimetic activity:

R′″ comprises H, halogen, C(halogen)₃, lower alkyl or alkoxy;

R″″ comprises H, halogen, C(halogen)₃, lower alkyl or alkoxy; and

R″ comprises —Y-D₁-D₂-T₂,

Y is optionally present and if present comprises C═CH or C≡C,

D₁ is optionally present and if present comprises alkyl,

D₂ comprises H, alkyl, NH, N-alkyl, O-alkyl, S-alkyl, a carbocyclic ring, a bicyclic ring, a tricyclic ring, an aromatic ring or a heteroaromatic ring,

T₂ is optionally present and if present comprises an aromatic ring, a heteroaromatic ring, a heterocyclic ring, H, OH, halogen or a substituent group.

In another advantageous variation R′″ comprises H, halogen, C(halogen)₃, lower alkyl or alkoxy;

R″″ comprises H, halogen, C(halogen)₃, lower alkyl or alkoxy; and

R″ comprises —Y-D₁-D₂-T₂,

Y comprises 0 to 1 of a carbocyclic ring having 4 to 6 ring members or a heterocyclic ring having 4 to 6 ring members with 1 or 2 heteroatoms.

D₁ is optionally present and if present comprises alkyl,

D₂ comprises H, alkyl, NH, N-alkyl, O-alkyl, S-alkyl, a carbocyclic ring, a bicyclic ring, a tricyclic ring, an aromatic ring or a heteroaromatic ring,

T₂ is optionally present and if present comprises an aromatic ring, a heteroaromatic ring, a heterocyclic ring, H, OH, halogen or a substituent group.

In one variation of the invention Ar comprises an aromatic ring having 5 or 6 ring members or a heteroaromatic ring having 5 or 6 ring members.

In another variation of the invention Ar comprises one of the structures:

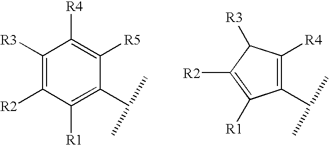

The Ar aromatic ring structure comprises 0 to 3 heteroatoms as ring members.

R1, R2, R3, R4 and R5, if present, each independently comprise H, OH, NH₂, halogen, N₃, NO₂, NCS, C(halogen)₃, CHO, OAc, OCH₃, OC₂H₅, CH₂OH, CH₂CH₂OH, CH₂CH₂CH₂OH, CN, C(═O)CH₃, COOH, COOCH₃, COOC₂H₅, COOCH(CH₃)₂, NHCOCH₃, SCH₃, SC₂H₅, NHCH₃, CH₂NH₂, CH₃, C₂H₅, C₃H₇, C₂H₃, ethynyl, alkoxy, alkylmercapto, alkylamino, di-alkylamino, alkylsulfinyl, alkylsulfonyl or methylene dioxy or other substituent groups as defined later

In another variation of the invention Ar comprises 1-, 2- or 3-pyrrolidinyl, 1-, 2-, 3- or 4-piperidinyl, 1-, 2- or 3-morpholinyl, 1-, 2- or 3-thiomorpholinyl, 1-, 2-, or 3- azetidinyl, 1-, or 2-piperazinyl, 2- or 3-tetrahydrofuranyl; or any above group substituted on any available ring carbon thereof by alkyl; or any above group unsubstituted on one or more nitrogen atoms, or any above group substituted on one or more nitrogen atoms independently by an alkyl, benzyl, lower-alkoxybenzyl or benzhydryl group; adamantyl; a carbocyclic ring, a substituted carbocyclic ring, a heteroaromatic ring, a substituted heteroaromatic ring, a heterocyclic ring, a substituted heterocyclic ring, a bicyclic ring, a substituted bicyclic ring, a heterobicyclic ring, a substituted heterobicyclic ring, a polycyclic ring, a substituted polycyclic ring, a heteropolycyclic ring or a substituted heteropolycyclic ring.

In an advantageous variation of the invention Ar comprises:

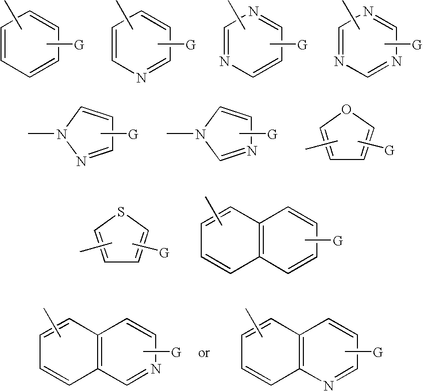

G comprises H, OH, NH₂, halogen, N₃, NO₂, NCS, CF₃, CHO, OAc, OCH₃, OC₂H₅, CH₂OH, CH₂CH₂OH, CH₂CH₂CH₂OH, CN, C(═O)CH₃, COOH, COOCH₃, COOC₂H₅, COOCH(CH₃)₂, NHCOCH₃, SCH₃, SC₂H₅, NHCH₃, CH₂NH₂, CH₃, C₂H₅, C₃H₇, C₂H₃, ethynyl, alkoxy, alkylmercapto, alkylamino, di-alkylamino, alkylsulfinyl, alkylsulfonyl or methylene dioxy.

Provisos with respect to compound formula I:

When Ar is 4-isopropyl pyridine or 4-isopropenyl pyridine; R′″ is hydrogen; and R″″ is hydrogen, then R″ can not be a straight or branched saturated alkyl having 1 to 20 carbon atoms.

When Ar is 4-isopropyl toluene or 4-isopropenyl toluene, and both R′″ and R″″ are hydrogen, R″ can not be a straight or branched saturated alkyl having 1 to 20 carbon atoms.

When R″ is C(CH₃)₂(CH₂)₅CH₃, R₂ and R₄ are methyl. R′ and R″ can not be H, OH or OCH₃.

Unless otherwise specifically defined, “acyl” refers to the general formula —C(O)alkyl.

Unless otherwise specifically defined, “acyloxy” refers to the general formula —O-acyl.

Unless otherwise specifically defined, “alcohol” refers to the general formula alkyl-OH and includes primary, secondary and tertiary variations.

Unless otherwise specifically defined, “alkyl” or “lower alkyl” refers to a linear, branched or cyclic alkyl group having from 1 to about 16 carbon atoms including, for example, methyl, ethyl, propyl, butyl, hexyl, octyl, isopropyl, isobutyl, tert-butyl, cyclopropyl, cyclohexyl, cyclooctyl, vinyl and allyl. Unless otherwise specifically defined, an alkyl group can be saturated or unsaturated. Unless otherwise specifically limited an alkyl group can be unsubstituted, singly substituted, or multiply substituted, with substituent groups in any possible position. Unless otherwise specifically limited, a cyclic alkyl group may include monocyclic, bicyclic, tricyclic, tetracyclic and polycyclic rings, for example norbornyl, adamantyl and related terpenes.

Unless otherwise specifically defined, “alkoxy” refers to the general formula —O-alkyl.

Unless otherwise specifically defined, “alkylmercapto” refers to the general formula —S-alkyl.

Unless otherwise specifically defined, “alkylamino” refers to the general formula —(NH)-alkyl.

Unless otherwise specifically defined, “di-alkylamino” refers to the general formula —N-(alkyl)₂. Unless otherwise specifically limited di-alkylamino includes cyclic amine compounds such as piperidine and morpholine.

Unless otherwise specifically defined, an aromatic ring is an unsaturated ring structure having about 5 to about 7 ring members and including only carbon as ring atoms. Unless otherwise specifically defined, an aromatic ring can be unsubstituted, singly substituted, or multiply substituted, with substituent groups in any possible position.

Unless otherwise specifically defined, “aryl” refers to an aromatic ring system that includes only carbon as ring atoms, for example phenyl, biphenyl or naphthyl. Unless otherwise specifically limited an aryl moiety can be unsubstituted, singly substituted, or multiply substituted, with substituent groups in any possible position.

Unless otherwise specifically defined, “aroyl” refers to the general formula —C(═O)-aryl.

Unless otherwise specifically defined, a bicyclic ring structure comprises 2 fused or bridged rings that include only carbon as ring atoms. The bicyclic ring structure may be saturated or unsaturated. Unless otherwise specifically limited a bicyclic ring structure can be unsubstituted, singly substituted, or multiply substituted, with substituent groups in any possible position. The individual rings may or may not be of the same type. Examples of bicyclic ring structures include, Dimethyl-bicyclo[3,1,1] heptane, bicyclo[2,2,1]heptadiene, decahydro-naphthalene and bicyclooctane.

Unless otherwise specifically defined, a carbocyclic ring is a non-aromatic ring structure having about 3 to about 8 ring members, substituted or unsubstituted, that includes only carbon as ring atoms, for example, cyclohexadiene or cyclohexane. Unless otherwise specifically limited a carbocyclic ring structure can be unsubstituted, singly substituted, or multiply substituted, with substituent groups in any possible position.

Unless otherwise specifically defined, “halogen” refers to an atom selected from fluorine, chlorine, bromine and iodine.

Unless otherwise specifically defined, a heteroaromatic ring is an unsaturated ring structure having about 5 to about 8 ring members that has carbon atoms and one or more heteroatoms, including oxygen, nitrogen and/or sulfur, as ring atoms, for example, pyridine, furan, quinoline, and their derivatives. Unless otherwise specifically limited a heteroaromatic ring can be unsubstituted, singly substituted, or multiply substituted, with substituent groups in any possible position.

Unless otherwise specifically defined, a heterobicyclic ring structure comprises 2 fused or bridged rings that include carbon and one or more heteroatoms, including oxygen, nitrogen and/or sulfur, as ring atoms. The heterobicyclic ring structure is saturated or unsaturated. The heterobicyclic ring structure can be unsubstituted, singly substituted, or multiply substituted, with substituent groups in any possible position. The individual rings may or may not be of the same type. Examples of heterobicyclic ring structures include tropane, quinuclidine and tetrahydro-benzofuran. Unless otherwise specifically defined, a heterocyclic ring is a saturated ring structure having about 3 to about 8 ring members that has carbon atoms and one or more heteroatoms, including oxygen, nitrogen and/or sulfur, as ring atoms, for example, piperidine, morpholine, piperazine, pyrrolidine, thiomorpholine, tetrahydropyridine, and their derivatives. The heterocyclic ring can be unsubstituted, singly substituted, or multiply substituted, with substituent groups in any possible position.

Unless otherwise specifically defined, a heterotricyclic ring structure comprises 3 rings that may be fused, bridged or both, and that include carbon and one or more heteroatoms, including oxygen, nitrogen and/or sulfur, as ring atoms. The heterotricyclic ring structure can be saturated or unsaturated. The heterotricyclic ring structure can be unsubstituted, singly substituted, or multiply substituted, with substituent groups in any possible position. The individual rings may or may not be of the same type. Examples of heterotricyclic ring structures include 2,4,10-trioxaadamantane, tetradecahydro-phenanthroline.

Unless otherwise specifically defined, a heteropolycyclic ring structure comprises more than 3 rings that may be fused, bridged or both and that includes carbon and one or more heteroatoms, including oxygen, nitrogen and/or sulfur, as ring atoms. The heteropolycyclic ring structure can be saturated or unsaturated. The heteropolycyclic ring structure can be unsubstituted, singly substituted or, if possible, multiply substituted, with substituent groups in any possible position. The individual rings may or may not be of the same type. Examples of heteropolycyclic ring structures include azaadamantine, 5-norbornene-2,3-dicarboximide.

Unless otherwise specifically defined, the term “phenacyl” refers to the general formula -phenyl-acyl.

Unless otherwise specifically defined, a polycyclic ring structure comprises more than 3 rings that may be fused, bridged or both fused and bridged and that includes carbon as ring atoms. The polycyclic ring structure can be saturated or unsaturated. Unless otherwise specifically limited a polycyclic ring structure can be unsubstituted, singly substituted, or multiply substituted, with substituent groups in any possible position. The individual rings may or may not be of the same type. Examples of polycyclic ring structures include adamantine, bicyclooctane, norbornane and bicyclononanes.

Unless otherwise specifically defined, a spirocycle refers to a ring system wherein a single atom is the only common member of two rings. A spirocycle can comprise a saturated carbocyclic ring comprising about 3 to about 8 ring members, a heterocyclic ring comprising about 3 to about 8 ring atoms wherein up to about 3 ring atoms may be N, S, or O or a combination thereof.

Unless otherwise specifically defined, a tricyclic ring structure comprises 3 rings that may be fused, bridged or both fused and bridged and that includes carbon as ring atoms. The tricyclic ring structure can be saturated or unsaturated. The tricyclic ring structure can be unsubstituted, singly substituted, or if possible, multiply substituted, with substituent groups in any possible position. The individual rings may or may not be of the same type. Examples of tricyclic ring structures include fluorene and anthracene.

Unless otherwise specifically limited the term substituted means substituted by at least one below described substituent group in any possible position or positions. Substituent groups for the above moieties useful in the invention are those groups that do not significantly diminish the biological activity of the inventive compound. Substituent groups that do not significantly diminish the biological activity of the inventive compound include, for example, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, COOX₃, SO₃H, PO₃H₂, SO₂NX₁X₂, CONX₁X₂, alkyl, alcohol, alkoxy, alkylmercapto, alkylamino, di-alkylamino, sulfonamide or thioalkoxy wherein X₁ and X₂ each independently comprise H or alkyl, or X₁ and X₂ together comprise part of a heterocyclic ring having about 4 to about 7 ring members and optionally one additional heteroatom selected from O, N or S, or X₁ and X2 together comprise part of an imide ring having about 5 to about 6 members and X₃ comprises H, alkyl, loweralkylhydroxy, or alkyl-NX₁X₂. Unless otherwise specifically limited, a substituent group may be in any possible position or any possible positions if multiply substituted.

Some of the inventive biphenyl and biphenyl-like cannabinoid compounds exhibit high affinity for the CB1 and/or CB2 cannabinoid receptors. Thus, another aspect of the invention is use of at least one of the inventive compounds to interact with cannabinoid receptors.

Further, some of the inventive biphenyl and biphenyl-like cannabinoid compounds show a very high selectivity for one of the cannabinoid receptors. These inventive selective compounds are able to interact with one cannabinoid receptor, for example the CB2 cannabinoid receptor, without affecting the other cannabinoid receptor to the same degree. Therefore, still another aspect of the invention is use of at least one of the inventive compounds to preferentially interact with one cannabinoid receptor.

Some of the inventive biphenyl and biphenyl-like cannabinoid compounds can act as high affinity modulators for cannabinoid receptors. The inventive cannabinoid compounds therefore are potential therapeutic agents through the modulation of the CB1 and/or CB2 cannabinoid receptors.

Some of the inventive biphenyl and biphenyl-like cannabinoid compounds described herein may be cannabinoid receptor agonists. The inventive cannabinoid agonists interact with the CB1 and/or CB2 cannabinoid receptor binding site to initiate a physiological or a pharmacological response characteristic of that receptor. Therefore, a further aspect of the invention is use of at least one of the inventive compounds to initiate an agonistic response from a cannabinoid receptor.

Some of the inventive biphenyl and biphenyl-like cannabinoid compounds described herein may be cannabinoid receptor antagonists. The inventive cannabinoid antagonists interact with the CB1 and/or CB2 cannabinoid receptor binding site to block other ligands from the receptor binding site without initiating a physiological or a pharmacological response characteristic of that receptor. Thus, cannabinoid antagonists typically oppose the cannabinoid receptor site response characteristics initiated by cannabinoid agonists. Therefore, a further aspect of the invention is use of at least one of the inventive compounds to oppose initiation of an agonistic response from a cannabinoid receptor.

The inventive biphenyl and biphenyl-like cannabinoid compounds described herein, and physiologically acceptable salts thereof, have pharmacological properties when administered in therapeutically effective amounts for providing a physiological response in individuals and/or animals. Thus, another aspect of the invention is the administration of a therapeutically effective amount of at least one of the inventive compounds, or a physiologically acceptable salt thereof, to an individual or animal to provide a physiological response.

Some of the novel biphenyl and biphenyl-like compounds in this invention are also more polar (less lipophilic) than known cannabinoids, a property that may help to improve their therapeutic usefulness in certain applications.

The novel biphenyl and biphenyl-like cannabinoids described herein, and physiologically acceptable salts thereof, have pharmacological properties when administered in therapeutically effective amounts for providing a physiological effect useful to treat central and peripheral pain, neuropathy, neurodegenerative diseases including multiple sclerosis, Parkinson's disease, Huntington's chorea, Alzheimer's disease; mental disorders such as schizophrenia and depression; to prevent or reduce endotoxic shock and hypotensive shock; to modulate appetite; to modulate the immune system; to reduce fertility; to prevent or reduce diseases associated with motor function such as Tourette's syndrome; to prevent or reduce inflammation; to provide neuroprotection and to suppress memory and produce peripheral vasodilation; to treat epilepsy, glaucoma, nausea associated with cancer chemotherapy and AIDS wasting syndrome as well as other ailments in which cannabinoid system is implicated. Thus, the invention involves the administration of a therapeutically effective amount of an inventive compound, or a physiologically acceptable salt thereof, to an individual or animal to provide a physiological effect.

The inventive compounds include any and all isomers and steroisomers. In general, the compositions of the invention may be alternately formulated to comprise, consist of, or consist essentially of, any appropriate components herein disclosed. The compositions of the invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

A better understanding of the invention will be obtained from the following detailed description of the presently preferred, albeit illustrative, embodiments of the invention.

### DESCRIPTION OF SOME PREFERRED EMBODIMENTS

As used herein a “therapeutically effective amount” of a compound, is the quantity of a compound which, when administered to an individual or animal, results in a sufficiently high level of that compound in the individual or animal to cause a discernible increase or decrease in stimulation of cannabinoid receptors. Physiological effects that result from cannabinoid receptor stimulation include analgesia, decreased nausea resulting from chemotherapy, sedation and increased appetite. Other physiological effects that result from cannabinoid receptor stimulation include relieving intraocular pressure in glaucoma patients and suppression of the immune system. Typically, a “therapeutically effective amount” of the compound ranges from about 10 mg/day to about 1,000 mg/day.

As used herein, an “individual” refers to a human. An “animal” refers to, for example, veterinary animals, such as dogs, cats, horses and the like, and farm animals, such as cows, pigs and the like.

The compound of the present invention can be administered by a variety of known methods, including orally, rectally, or by parenteral routes (e.g., intramuscular, intravenous, subcutaneous, nasal or topical). The form in which the compounds are administered will be determined by the route of administration. Such forms include, but are not limited to, capsular and tablet formulations (for oral and rectal administration), liquid formulations (for oral, intravenous, intramuscular, subcutaneous ocular, intranasal, inhalation based or transdermal administration) and slow releasing microcarriers (for rectal, intramuscular or intravenous administration). The formulations can also contain a physiologically acceptable vehicle and optional adjuvants, flavorings, colorants and preservatives. Suitable physiologically acceptable vehicles may include, for example, saline, sterile water, Ringer's solution and isotonic sodium chloride solutions. The specific dosage level of active ingredient will depend upon a number of factors, including, for example, biological activity of the particular preparation, age, body weight, sex and general health of the individual being treated.

The following examples are given for purposes of illustration only in order that the present invention may be more fully understood. These examples are not intended to limit in any way the scope of the invention unless otherwise specifically indicated.

### EXAMPLES

The inventive compounds are generally represented by compound formula I and include physiologically acceptable salts thereof.

A number of different biphenyl cannabinoids were prepared. Biphenyl cannabinoids synthesized with different functional groups are depicted in Table 1.

TABLE 1

| | |
|---|---|
| 1 | 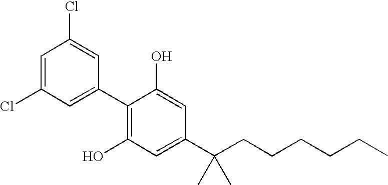 |
| 2 | 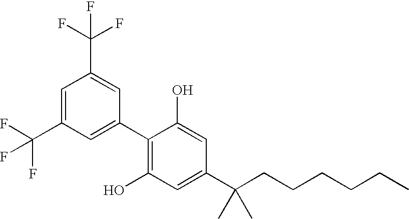 |
| 3 | 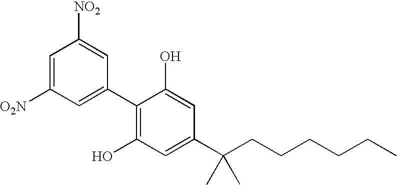 |
| 4 | 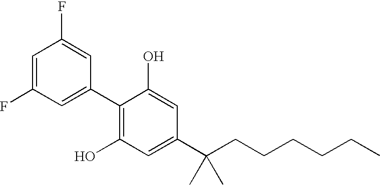 |
| 5 | 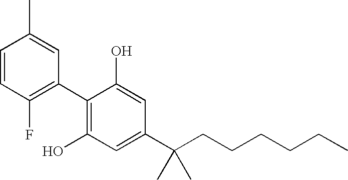 |
| 6 | 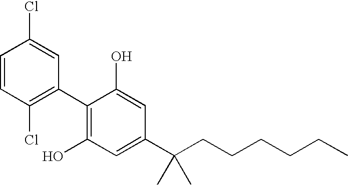 |
| 7 | 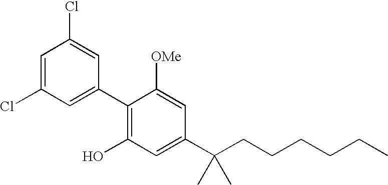 |
| 8 | 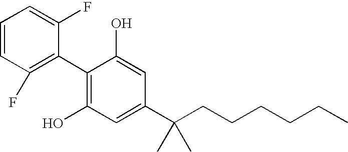 |
| 9 | 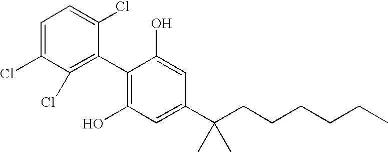 |
| 10 | 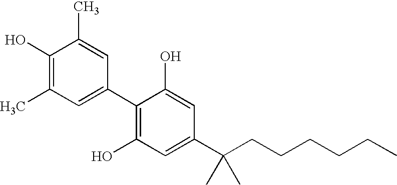 |
| 11 | 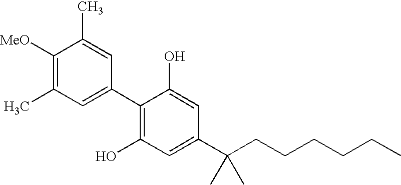 |
| 12 | 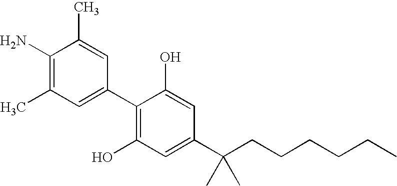 |
| 13 | 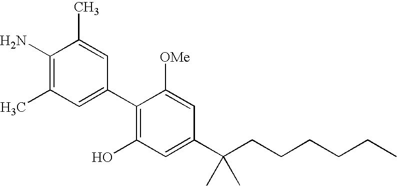 |
| 14 | 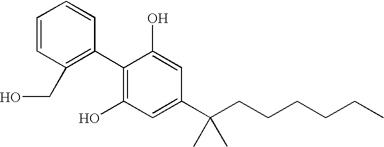 |
| 15 | 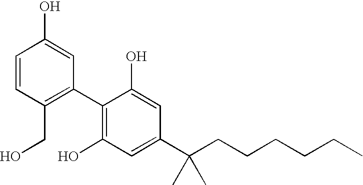 |
| 16 | 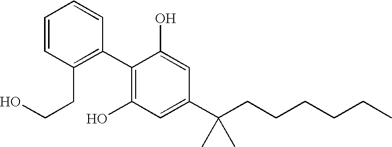 |
| 17 | 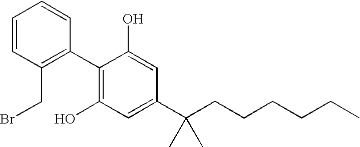 |
| 18 | 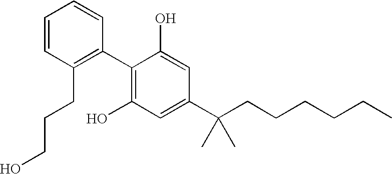 |
| 19 | 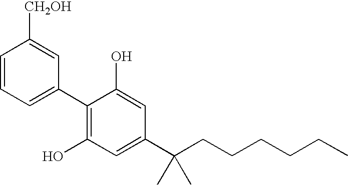 |
| 20 | 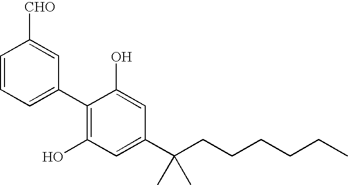 |
| 21 | 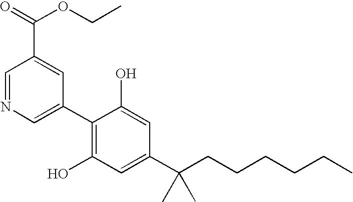 |
| 22 | 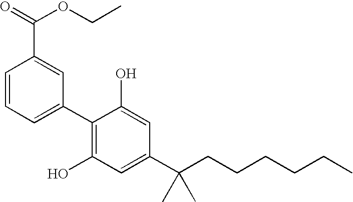 |
| 23 | 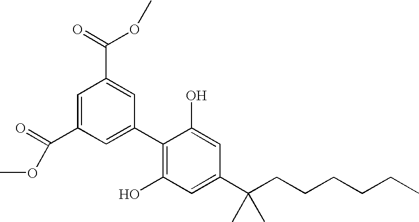 |
| 24 | 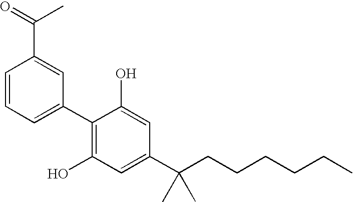 |
| 25 | 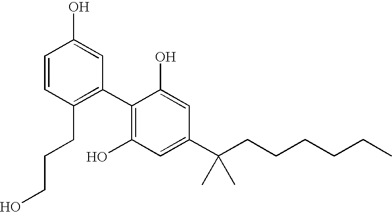 |
| 26 | 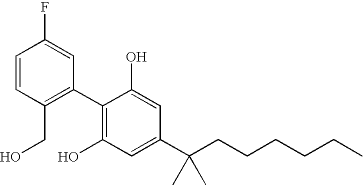 |
| 27 | 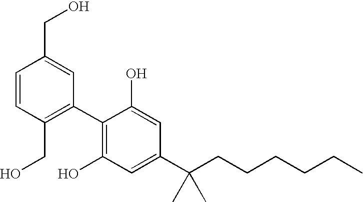 |
| 28 | 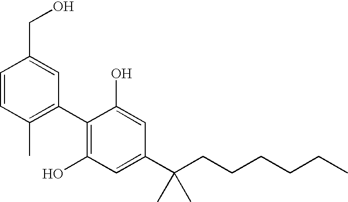 |
| 29 | 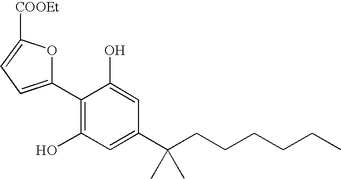 |
| 30 | 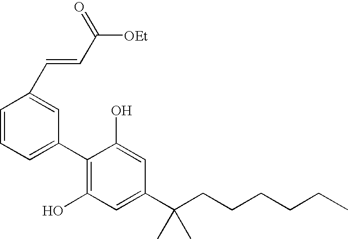 |
| 31 | 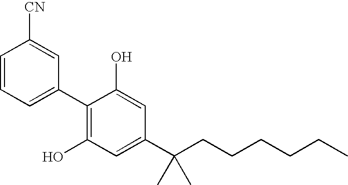 |
| 32 | 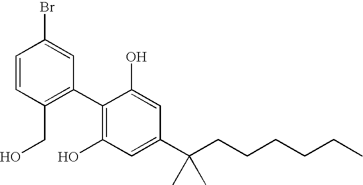 |
| 33 | 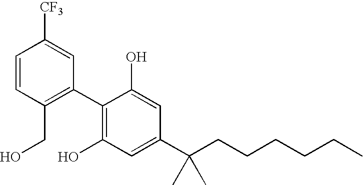 |
| 34 | 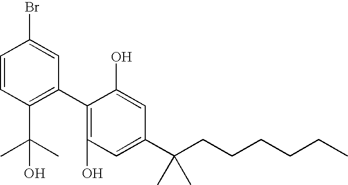 |
| 35 | 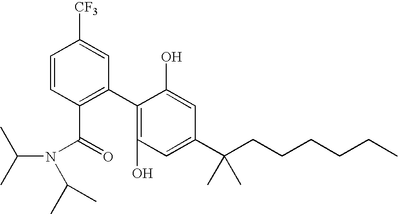 |
| 36 | 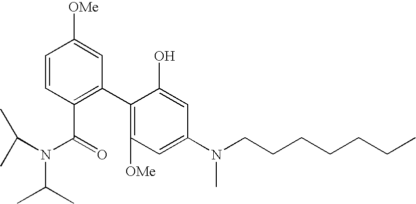 |
| 37 | 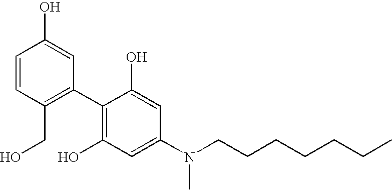 |
| 38 | 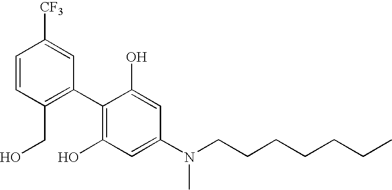 |

Some inventive analogs were tested for CB2 receptor binding affinity and for CB1 receptor affinity (to determine selectivity). As used herein, “binding affinity” is represented by the Kᵢ value which is the inhibition constant correlated with the concentration of an analog required to occupy the 50% of the total number (Bmax) of the receptors. The lower the Kᵢ value, the higher the binding affinity. As used herein an analog is said to have “binding selectivity” if it has higher binding affinity for one receptor compared to the other receptor; e.g. a cannabinoid analog which has an Kᵢ of 0.1 nM for CB2 and 10 nM for CB1, is 100 times more selective for the CB2 receptor. For the CB1 receptor binding studies, membranes were prepared from rat forebrain membranes according to the procedure of P. R. Dodd et al, A Rapid Method for Preparing Synaptosomes: Comparison with Alternative Procedures, Brain Res., 107-118 (1981). The binding of the novel analogues to the CB1 cannabinoid receptor was assessed as described in W. A. Devane et al, Determination and Characterization of a Cannabinoid Receptor in a Rat Brain, Mol. Pharmacol., 34, 605-613 (1988) and A. Charalambous et al, 5′-azido Δ⁸-THC: A Novel Photoaffinity Label for the Cannabinoid Receptor, J. Med. Chem., 35, 3076-3079 (1992) with the following changes. The above articles are incorporated by reference herein.

Membranes, previously frozen at −80° C., were thawed on ice. To the stirred suspension was added three volumes of TME (25 mM Tris-HCl buffer, 5 mM MgCl₂ and 1 mM EDTA) at a pH 7.4. The suspension was incubated at 4° C. for 30 min. At the end of the incubation, the membranes were pelleted and washed three times with TME.

The treated membranes were subsequently used in the binding assay described below. Approximately 30 μg of membranes were incubated in silanized 96-well microtiter plate with TME containing 0.1% essentially fatty acid-free bovine serum albumin (BSA), 0.8 nM [³H] CP-55,940, and various concentrations of test materials in a final volume of 200 μL. The assays were incubated for 1 hour at 30° C. and then immediately filtered using Packard Filtermate 196 harvester and Whatman GF/C filterplates and washed with wash buffer (TME) containing 0.5% BSA. Radioactivity was detected using MicroScint 20 scintillation cocktail added directly to the dried filterplates, and the filterplates were counted using a Packard Instruments Top-Count. Nonspecific binding was assessed using 100 nM CP-55,940. Data collected from three independent experiments performed with duplicate determinations was normalized between 100% and 0% specific binding for [³H] CP-55,940, determined using buffer and 100 nM CP-55,940. The normalized data was analyzed using a 4-parameter nonlinear logistic equation to yield IC₅₀ values. Data from at least two independent experiments performed in duplicate was used to calculate IC₅₀ values which were converted to Kᵢ values using the assumptions of Cheng et al, Relationship Between the Inhibition Constant (Kᵢ) and the concentration of Inhibitor which causes 50% Inhibition (IC₅₀) of an Enzymatic Reaction, Biochem. Pharmacol., 22, 3099-3102, (1973), which is incorporated by reference herein.

For the CB2 receptor binding studies, membranes were prepared from frozen mouse spleen essentially according to the procedure of P. R. Dodd et al, A Rapid Method for Preparing Synaptosomes: Comparison with Alternative Procedures, Brain Res., 226, 107-118 (1981) which is incorporated by reference herein. Silanized centrifuge tubes were used throughout to minimize receptor loss due to adsorption. The CB2 binding assay was conducted in the same manner as for the CB1 binding assay. The binding affinities (Kᵢ) were also expressed in nanomoles (nM). TABLE 2 lists binding affinities for some of the inventive compounds.

TABLE 2

| Compound | CB1 (nM) | CB2 (nM) |
|---|---|---|
| 1 | 2.6 | 0.6 |
| 2 | 9.7 | 0.9 |
| 3 | 198.7 | 3.5 |
| 4 | 57.5 | 8.4 |
| 5 | 6.5 | 2.3 |
| 6 | 7.7 | 2.6 |
| 7 | 104.8 | 13.9 |
| 8 | 40.3 | 13.4 |
| 9 | 23.2 | 3.4 |
| 10 | 1365 | 15.3 |
| 11 | 1080 | 12.8 |
| 12 | 796.7 | 8.0 |
| 14 | 53.8 | 1.4 |
| 15 | 140.8 | 4.5 |
| 21 | 17.2 | 0.2 |
| 22 | 241.0 | 0.8 |
| 24 | 297.4 | 11.3 |
| 25 | 12.2 | 1.0 |
| 26 | 24.5 | 1.8 |
| 27 | 29.6 | 2.6 |
| 28 | 70.4 | 7.0 |
| 29 | 3223 | 35.4 |
| 30 | 397.0 | 10.0 |
| 31 | 20 | 1.1 |
| 32 | 8.6 | 0.7 |
| 36 | 1875.0 | 154.0 |

### Typical Preparation Procedures

The inventive compounds were prepared by the Suzuki Coupling (Scheme 1 and 2). The following examples are given for purposes of illustration only in order that the present invention may be more fully understood. These examples are not intended to limit in any way to the practice of the invention.

The preparation procedures include aspects of the following references, the disclosures of which are hereby incorporated by reference. Alo, B. I.; Kandil, A.; Patil, P. A.; Sharp, M. J.; Siddiqui, M. A.; and Snieckus, V. Sequential Directed Ortho Metalation-Boronic Acid Cross-Coupling Reactions. A general Regiospecific Route to Oxygenerated Dibenzo[b,d]pyran-6-ones Related to Ellagic Acid, J. Org. Chem. 1991, 56, 3763-3768. Watanabe, T.; Miyaura, N.; Suzuki, A., Synthesis of Sterically Hindered Biaryls via the Palladium Catalyzed Cross-Couplinq Reaction of Arylboronic Acids or their Esters with Haloarenes, Synlett 1992, 207-210. Morris, S,; Mechoulam, R.; and Irene, Y., Halogenation of phenols and Phenyl ethers with Potassium Halides in the Presence of 18-Crown-6 on Oxidation with m-Chloroperbenzoic Acid, J. Chem. Soc., Perkin Trans. 1 1987, 1423-1427. Gareau, Y.; Dufresne, C.; Gallant, M.; Rochette, C.; Sawyer, N.; Slipetz, D. M.; Tremblay, N.; Weech, P. K.; Metters, K. M.; Labelle, M. Structure activity relationships of tetrahydrocanabinol analogs on human cannabinoid receptors. Bioorg. Med. Chem. Lett. 1996, 6(2), 89-94. Beak, P.; and Brown, R A., The Tertiary Amide as an Effective Director of Ortho Lithiation, J. Org. Chem. 1982, 47, 34-36. Rhee, M. H.; Vogel, Z.; Barg, J.; Bayewitch, M.; Levy, R.; Hanus, L.; Breuer, A.; and Mechoulam, R., Cannabinol Derivatives: Binding to Cannabinoid Receptors and Inhibition of Adenylcyclase, J. Med. Chem. 1997, 40, 3228-3233. Fahrenholtz, K. E., Lurie, M. and Kierstead, A R. W., The Total Synthesis of dl-Δ⁹-Tetrahydrocannabinol and Four of Its Isomers, J. Amer. Chem. Soc. 1967, 89:23, 5934-5941. Love, R. Bender, P. E., Dowalo, F., Macko, E., and Fowler, P., Cannabinoids. Structure-Activity Studies Related to 1,2-Dimethylheptyl Derivatives, J. Med. Chem 1973, 16,1200-1206.

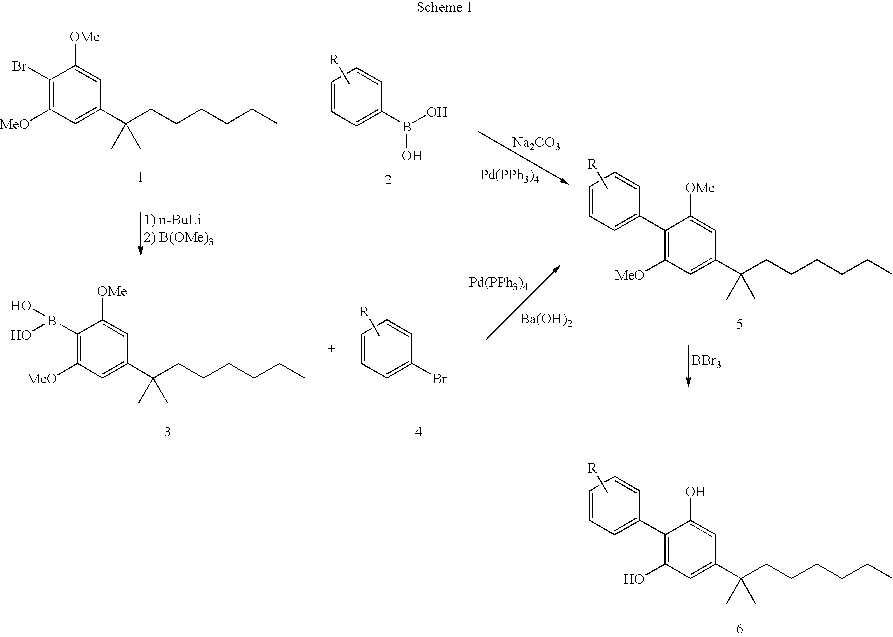

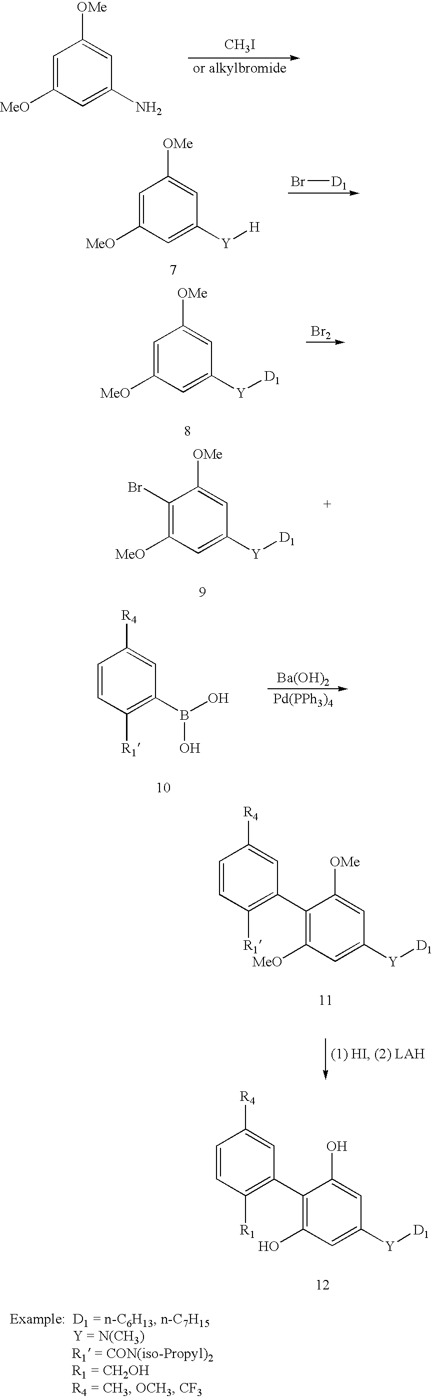

General. Proton NMR spectrums were recorded on Bruker 200 MHz and 500 MHz spectrometers as solutions in deuterated chloroform or other suitable solvents. Routine GC-MS analyses of the intermediates and the final products were performed on a Hewlett-Packard 6890A series gas chromatograph coupled with a mass selective detector (MSD). Anhydrous tetrahydrofuran (THF) and anhydrous ethylene glycol dimethyl ether (dimethoxyethane, or DME) were purchased from the Aldrich Chemical Company. DME was degassed using argon for the Suzuki coupling reaction. Trimethylborate, tetrakis(triphenylphosphine)palladium, barium hydroxide octahydrate, boron tribromide, boron triiodide, sodium carbonate and n-butyllithium were also purchased from the Aldrich Chemical Company. Purification by flash chromatograph was carried out on silica gel, grade 9385 (230-400 mesh) using solvents indicated in the parenthesis as eluents. Thin layer chromatographic analyses were carried out on Whatman 60F₂₅₄ polyester plates.

As indicated in Scheme 1, the common intermediate 5 can be synthesized by the Suzuki coupling reaction, either from aryl bromide 1 and commercially available boronic acid 2 or from aryl boronic acid 3 and widely commercially available aryl bromide 4. Multiply substituted aryl boronic acid or multiply substituted aryl bromide can be used in Scheme 1 to prepare the inventive compounds having multiply substituted Ar rings.

### General Procedure for the Synthesis of Biaryl Dimethoxyether 5 from the Commercial Available Aryl Boronic Acid 2

To a suspension of Pd(PPh₃)₄ (0.05 Equiv) in anhydrous DME was added the arylbromide 1 and the mixture was stirred for 10 min at room temperature. To this solution were added sequentially the arylboronic acid 2 (1.5 equiv) in a minimum of EtOH and aqueous Na₂CO₃ (2 M solution, 2.0 equiv), and the mixture was refluxed for 18 hour, cooled, and subjected to filtration through a short silica gel. The filtrate was treated with saturated NaCl solution, dried (Na₂SO₄) and evaporated. The residue was purified by flash column chromatography on silica gel with petroleum ether/acetone (100:1.5˜2) to afford the Biaryl dimethoxyether 5.

### Intermediate 3

A solution of 1 (2.78 g, 8 mmol) in 8 ml of THF was stirred and cooled to −78° C. under N₂. The solution (1.6 M in Hexane) of n-BuLi (5.5 ml, 8.8 mmol, 1.1 equiv) was added dropwise over 30 min. The reaction mixture was stirred for an additional 15 min at −78° C., treated with B(OMe)₃ (2.7 ml, 24 mmol, 3 equiv) and allowed to warm to room temperature over 12 h. it was cooled 0° C. and acidified to pH 6.5 with 5% aqueous HCl, and extracted with methylene chloride. The organic layer was washed with saturated brine solution, dried (Na₂SO₄) and evaporated. The residue was purified by flash column chromatography on silica gel with petroleum ether/acetone (100:12) to afford the colorless solid Intermediate 3 (2.1 g, 85% yield).

### General Procedure for the Synthesis of Biaryl Dimethoxyether 5 from the Commercial Available Aryl Halide 4

To a flask equipped with a reflux condenser, a septum inlet, and a magnetic stirring bar, were added Pd(PPh₃)₄ (0.05 Equiv), the boronic acid 3 (1 equiv), and Ba(OH)₂.8H₂O (1.5 equiv). The flask was flushed with Nitrogen and charged with DME (6 ml/mmol 3), H₂O (1 ml/mmol 3) and aryl halide 4 (1.2 equiv) through the septum inlet with a syringe. The mixture was heated in an oil bath at 80° C. with stirring untill the boronic acid 3 could no longer be detected in the reaction mixture. Subsequently, the reaction mixture was cooled, and subjected to filtration through a short silica gel. The filtrate was treated with saturated NaCl solution, dried (Na₂SO₄) and evaporated. The residue was purified by flash column chromatography on silica gel with petroleum ether/acetone (100:1.5˜2) to afford the biaryl dimethoxyether 5.

### General Procedure for the Synthesis of Biaryl Compounds

A solution of the biaryl dimethoxyether 5 (0.25 M) in CH₂Cl₂ was stirred and cooled in an ice bath. Boron tribromide (2.5 Equiv, 1 M in CH₂Cl₂) was added dropwise. The mixture was stirred at 0° C. for 4 h and the reaction was quenched by adding H₂O slowly at 0° C. The mixture was then diluted with ether, washed (saturated NaCl), dried (Na₂SO₄) and evaporated. The residue was purified by flash column chromatography on silica gel with petroleum ether/acetone (100:12˜20) to afford the Biaryl 6.

The synthesis of compound 12 represented biphenyls was carried out by Suzuki coupling via an ortho-amide facilitated reaction as described by Watanabe, T.; Miyaura, N.; and Suzuki, A., in Synthesis of Sterically Hindered Biaryls via the Palladium Catalyzed Cross-Coupling Reaction of Arylboronic Acids or their Esters with Haloarenes, Synlett 1992, 207-210.

Those skilled in the art will recognize, or be able to ascertain with no more than routine experimentation, many equivalents to the specific embodiments of the invention disclosed herein. Such equivalents are intended to be encompassed by the scope of the invention.

What is claimed is:

1. A compound of formula I below, and physiologically acceptable salts, comprising: 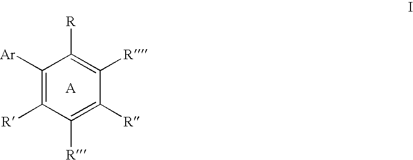
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R″ can not be H, OH or OCH₃.

2. The compound of claim 1 wherein only one of R″, R′″ and R″″ comprises Y-D₁-D₂-T₂ and the others of R″, R′″ and R″″ each independently comprise H, halogen, alkyl, alkoxy or a substituent group.

3. The compound of claim 1 wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises C(CH₃)₂, CH₂ or CH(CH₃),
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

4. The compound of claim 1 wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises O, NH or N-alkyl,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

5. The compound of claim 1 wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y is optionally present and if present comprises C═CH or C═C,
D₁ is optionally present and if present comprises alkyl,
D₂ comprises H, alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

6. The compound of claim 1 wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises 0 to 1 of a carbocyclic ring having 4 to 6 ring members or a heterocyclic ring having 4 to 6 ring members with 1 or 2 heteroatoms.
D₁ is optionally present and if present comprises alkyl,
D₂ comprises H, alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

7. The compound of claim 1 wherein Ar comprises an aromatic ring having 5 or 6 ring members or a heteroaromatic ring having 5 or 6 ring members.

8. The compound of claim 1 wherein Ar comprises one of the structures: 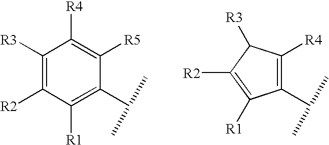
and,
the Ar aromatic ring structure comprises 0 to 3 heteroatoms as ring members;
R1, R2, R3, R4 and R5 each independently comprise H, OH, NH₂, halogen, N₃, NO₂, NCS, C(halogen)₃, CHO, OAc, OCH₃, OC₂H₅, CH₂OH, CH₂CH₂OH, CH₂CH₂CH₂OH, CN, C(═O)CH₃, COOH, COOCH₃, COOC₂H₅, COOCH(CH₃)₂, NHCOCH₃, SCH₃, SC₂H₅, NHCH₃, CH₂NH₂, CH₃, C₂H₅, C₃H₇, C₂H₃, ethynyl, alkoxy, alkylmercapto, alkylamino, di-alkylamino, alkylsulfinyl, alkylsulfonyl or methylene dioxy or a substituent group.

9. The compound of claim 1 wherein Ar comprises 1-, 2- or 3-pyrrolidinyl, 1-, 2-, 3- or 4-piperidinyl, 1-, 2- or 3-morpholinyl, 1-, 2- or 3-thiomorpholinyl, 1-, 2- or 3- azetidinyl, 1-, or 2-piperazinyl, 2- or 3-tetrahydrofuranyl; or any above group substituted on any available ring carbon thereof by alkyl; or any above group unsubstituted on one or more nitrogen atoms, or any above group substituted on one or more nitrogen atoms independently by an alkyl, benzyl, lower-alkoxybenzyl or benzhydryl group; adamantyl; a carbocyclic ring, a substituted carbocyclic ring, a heteroaromatic ring, a substituted heteroaromatic ring, a heterocyclic ring, a substituted heterocyclic ring, a bicyclic ring, a substituted bicyclic ring, a heterobicyclic ring, a substituted heterobicyclic ring, a polycyclic ring, a substituted polycyclic ring, a heteropolycyclic ring or a substituted heteropolycyclic ring.

10. The compound of claim 1 wherein Ar comprises: 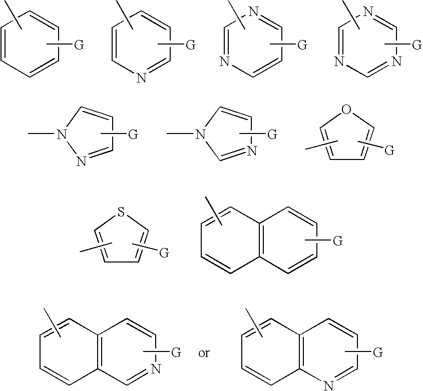
G comprises H, OH, NH₂, halogen, N₃, NO₂, NCS, CF₃, CHO, OAc, OCH₃, OC₂H₅, CH₂OH, CH₂CH₂OH, CH₂CH₂CH₂OH, CN, C(═O)CH₃, COOH, COOCH₃, COOC₂H₅, COOCH(CH₃)₂, NHCOCH₃, SCH₃, SC₂H₅, NHCH₃, CH₂NH₂, CH₃, C₂H₅, C₃H₇, C₂H₃, ethynyl, alkoxy, alkylmercapto, alkylamino, di-alkylamino, alkylsulfinyl, alkylsulfonyl or methylene dioxy.

11. A pharmaceutical preparation comprising a therapeutically effective amount of at least one compound of formula I below, and physiologically acceptable salts thereof: 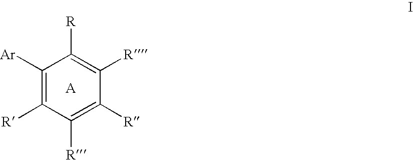
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R′″ can not be H, OH or OCH₃.

12. The pharmaceutical preparation of claim 11 wherein only one of R″, R′″ and R″″ comprises Y-D₁-D₂-T₂ and the others of R″, R′″ and R″″ each independently comprise H, halogen, alkyl, alkoxy or a substituent group.

13. The pharmaceutical preparation of claim 11, wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

14. A method of stimulating a cannabinoid receptor in an individual or animal comprising administering to the individual or animal a therapeutically effective amount of a therapeutically effective amount of at least one compound of formula I below, and physiologically acceptable salts thereof: 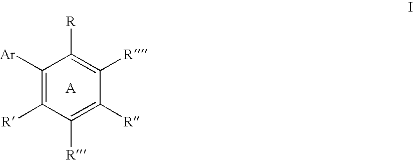
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R″ can not be H, OH or OCH₃.

15. The method of claim 14 wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

16. A method of selectively stimulating CB2 cannabinoid receptors in an individual or animal comprising administering to the individual or animal a therapeutically effective amount of at least one compound of formula I below, and physiologically acceptable salts thereof: 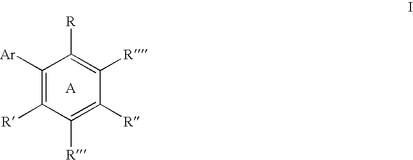
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R″ can not be H, OH or OCH₃.

17. The method of claim 16, wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

18. A method of treating a condition comprising administering to an individual or animal having the condition a therapeutically effective amount of at least one compound of formula I below, and physiologically acceptable salts thereof: 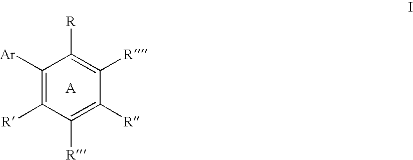
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R″ can not be H, OH or OCH₃.

19. The method of claim 18, wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

20. A method of providing a physiological response in an individual or animal comprising administering to the individual or animal a therapeutically effective amount of at least one compound of formula I below, and physiologically acceptable salts thereof: 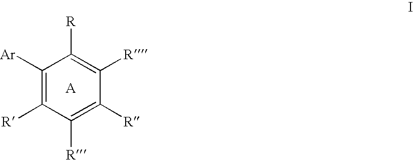
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R″ can not be H, OH or OCH₃.

21. The method of claim 20, wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.

22. A method of treating a condition selected from central and peripheral pain, neuropathy, neurodegenerative diseases including multiple sclerosis, Parkinson's disease, Huntington's chorea, Alzheimer's disease; mental disorders such as schizophrenia and depression, endotoxic shock, hypotensive shock; or of modulating appetite; or of modulating the immune system; or of reducing fertility; or of treating diseases associated with motor function such as Tourette's syndrome; or of treating inflammation; or of providing neuroprotection; or of suppressing memory; or of producing peripheral vasodilation; or of treating epilepsy, glaucoma, nausea associated with cancer chemotherapy or nausea associated with Aids wasting syndrome comprising administering to an individual or animal having the condition a therapeutically effective amount of at least one compound at least one compound of formula I below, and physiologically acceptable salts thereof: 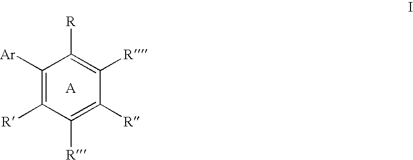
wherein,
the “A” ring atoms of compound formula I comprise carbon and 0 to 2 nitrogen heteroatoms;
Ar is an aromatic ring, an aromatic ring comprising at least one substituent group, a heteroaromatic ring, a heteroaromatic ring comprising 1 to 5 substituent groups, a heterocyclic ring or a heterocyclic ring comprising at least one substituent group;
R comprises H, OH, OCH₃, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R′ comprises H, OH, alkoxy, OCH₂CH₂OH, alcohol, NH₂, PO₃H, OPO₃H, OSO₃H, halogen, C(halogen)₃, SE₁, OE₁ or NE₁E₂,
E₁ and E₂ are each independently H or alkyl;
R″, R′″ and R″″ each independently comprises Y-D₁-D₂-T₂, H, halogen, alkyl, alkoxy or a substituent group,
Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, alkyl, NH, N-alkyl, O-alkyl, S-alkyl, a carbocyclic ring, a bicyclic, a tricyclic ring, an aromatic or heteroaromatic ring,
T₂ is optionally present and if present comprises an aromatic ring, a substituted aromatic ring, a heteroaromatic ring, a substituted heteroaromatic ring, a heterocyclic ring, a substituted heterocyclic ring, H, OH, halogen, or a substituent group;
with the proviso that,
when Ar is 4-isopropyl pyridine or 4-isopropenyl pyridine, R′″ is hydrogen, and R″″ is hydrogen, then R″ can not be a straight or branched saturated alkyl having 1 to 20 carbon atoms;
when Ar is 4-isopropyl toluene or 4-isopropenyl toluene, and both R′″ and R″″ are hydrogen, R″ can not be a straight or branched saturated alkyl having 1 to 20 carbon atoms;
when R″ is C(CH₃)₂(CH₂)₅CH₃, R₂ and R₄ are methyl, then R′ and R″ can not be H, OH or OCH₃.

23. The method of claim 22, wherein:
R′″ comprises H, halogen, C(halogen)₃, lower alkyl or alkoxy;
R″″ comprises H, halogen, C(halogen)₃, lower alkyl or alkoxy; and
R″ comprises —Y-D₁-D₂-T₂,
Y comprises Y is optionally present and if present comprises O, S, NH, N-alkyl, C═CH, C≡C, CH₂, CH(CH3), C(CH₃)₂, a carbocyclic ring having 4 to 6 ring members or a heterocyclic ring having 4 to 6 ring members with 1 or 2 heteroatoms,
D₁ is optionally present and if present comprises alkyl,
D₂ comprises H, an alkyl, NH, N-alkyl, O-alkyl, S-alkyl, a carbocyclic ring, a bicyclic ring, a tricyclic ring, an aromatic ring or a heteroaromatic ring,
T₂ is optionally present and if present comprises an aromatic ring, a heteroaromatic ring, a heterocyclic ring, H, OH, halogen or a substituent group.
